(19) United States
(12) Patent Application Publication
(10) Pub. No.: US 2022/0245471 A1
(43) Pub. Date: Aug. 4, 2022

(54) COMPUTER-READABLE RECORDING MEDIUM STORING GENERATION PROGRAM, GENERATION METHOD, AND GENERATION APPARATUS

(71) Applicant: FUJITSU LIMITED, Kawasaki-shi (JP)

(72) Inventor: Katsuhiko MURAKAMI, Yokohama (JP)

(73) Assignee: FUJITSU LIMITED, Kawasaki-shi (JP)

(21) Appl. No.: 17/553,794

(22) Filed: Dec. 17, 2021

(30) Foreign Application Priority Data
Feb. 1, 2021 (JP) 2021-014616

(51) Int. Cl. G06N 5/02; G16C 20/80; G16C 20/70; G16C 20/10

(52) U.S. Cl. CPC G06N 5/022; G16C 20/10; G16C 20/70; G16C 20/80

(57) ABSTRACT

A process includes acquiring a plurality of pieces of data each of which is data in which an element of a first item, an element of a second item, and a relation between the element of the first item and the element of the second item are associated with one another, transforming each of identical elements that appear in common as either item of the first item or the second item in two or more pieces of data among the acquired plurality of pieces of data, into a unique element in the plurality of pieces of data, and generating, based on the plurality of pieces of data resulting from the transforming, a graph that is constituted by a plurality of nodes each of which represents a different element and that indicates a relation between elements in a multidimensional vector space.

### CROSS-REFERENCE TO RELATED APPLICATION

This application is based upon and claims the benefit of priority of the prior Japanese Patent Application No. 2021-014616, filed on Feb. 1, 2021, the entire contents of which are incorporated herein by reference.

### FIELD

The embodiment discussed herein is related to a computer-readable recording medium storing a generation program, a generation method, and a generation apparatus.

### BACKGROUND

In the related art, there is a knowledge graph that is constituted by nodes representing elements and that indicates relations between the elements in a multidimensional vector space. For example, in the field of chemistry, a knowledge graph is constituted by nodes representing compound names, nodes representing molecular formulas or molecular weights of compounds, nodes representing functions or applications of compounds, or the like.

As related art, for example, there is a technique for generating negative statements from statements, generating candidate statements by combination of the statements, generating negative candidate statements by combination of the candidate statements with the negative statements, and scoring the candidate statements by using the candidate statements and the negative candidate statements in a relation learning model.

Japanese Laid-open Patent Publication No. 2018-206374 is disclosed as related art.

### SUMMARY

According to an aspect of the embodiments, a non-transitory computer-readable recording medium storing a generation program for causing a computer to execute a process, the process includes acquiring a plurality of pieces of data each of which is data in which an element of a first item, an element of a second item, and a relation between the element of the first item and the element of the second item are associated with one another, transforming each of identical elements that appear in common as either item of the first item or the second item in two or more pieces of data among the acquired plurality of pieces of data, into a unique element in the plurality of pieces of data, and generating, based on the plurality of pieces of data resulting from the transforming, a graph that is constituted by a plurality of nodes each of which represents a different element and that indicates a relation between elements in a multidimensional vector space.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is an explanatory diagram (part 1) illustrating an example of a generation method according to an embodiment;

FIG. 2 is an explanatory diagram (part 2) illustrating the example of the generation method according to the embodiment;

FIG. 3 is an explanatory diagram illustrating an example of an information processing system;

FIG. 4 is a block diagram illustrating an example of a hardware configuration of an information processing apparatus;

FIG. 5 is an explanatory diagram illustrating an example of stored content of a triple data management table;

FIG. 6 is an explanatory diagram illustrating an example of stored content of a knowledge graph management table;

FIG. 7 is a block diagram illustrating an example of a hardware configuration of a client apparatus;

FIG. 8 is a block diagram illustrating an example of a functional configuration of the information processing apparatus;

FIG. 9 is an explanatory diagram (part 1) illustrating an example of an operation of the information processing apparatus;

FIG. 10 is an explanatory diagram (part 2) illustrating the example of the operation of the information processing apparatus;

FIG. 11 is an explanatory diagram illustrating another example of the operation of the information processing apparatus;

FIG. 12 is an explanatory diagram (part 1) illustrating an actual application example of the information processing apparatus;

FIG. 13 is an explanatory diagram (part 2) illustrating the actual application example of the information processing apparatus;

FIG. 14 is a flowchart illustrating an example of a procedure of an overall process; and

FIG. 15 is a flowchart illustrating an example of a procedure of a determination process.

### DESCRIPTION OF EMBODIMENTS

In the related art, a graph that accurately indicates relations between elements may not be generated in some cases. For example, in a case where there are a plurality of relations between certain elements, nodes that represent the respective elements may not be arranged in a multidimensional vector space such that the individual relations are accurately indicated. Consequently, a graph that accurately indicates the relations between the elements may not be generated.

An embodiment of a technique that enables generation of a graph that accurately indicates relations between elements will be described in detail below with reference to the drawings.

[Example of Generation Method According to Embodiment]

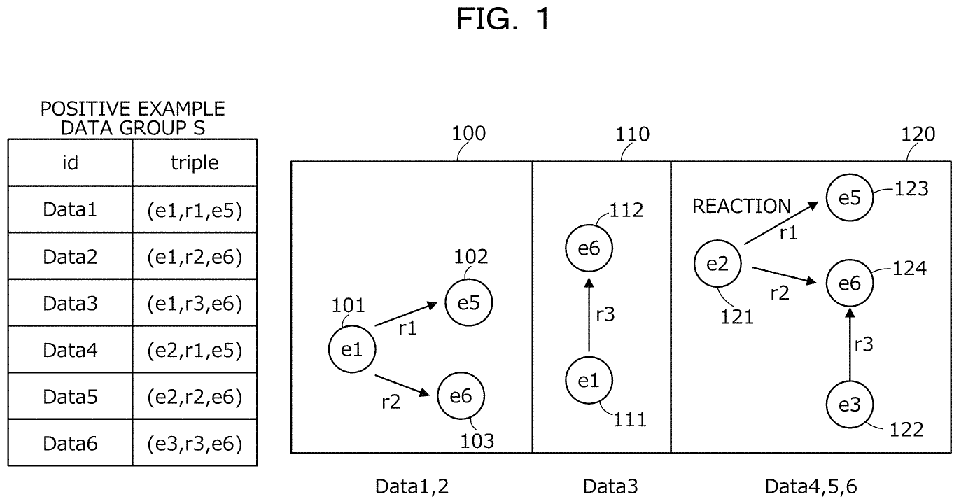

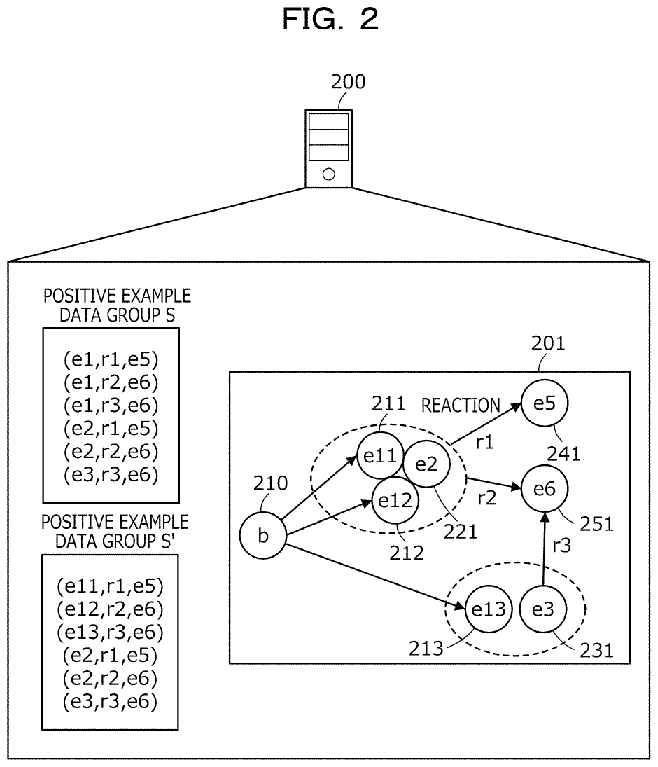

FIGS. 1 and 2 are explanatory diagrams illustrating an example of a generation method according to an embodiment. In the related art, there is a knowledge graph that is constituted by nodes representing elements and that indicates relations between the elements in a multidimensional vector space. For example, in the field of chemistry, a knowledge graph is constituted by nodes representing compound names, nodes representing molecular formulas or molecular weights of compounds, nodes representing functions or applications of compounds, or the like. The compounds are, for example, proteins or the like.

When a user desires to determine whether there is a predetermined relation between any elements, a knowledge graph may be used. For example, a knowledge graph is used in determining whether triple data, in which an element serving as “s” (subject), an element serving as “o” (object), and an element serving as “r” (predicate) indicating a relation between the element serving as “s” and the element serving as “o” are associated with one another, is correct answer data. The correct answer data is triple data in which an element serving as “r” correctly indicates a relation between an element serving as “s” and an element serving as “o”.

It is considered that the accuracy with which the user determines, by using the knowledge graph, whether the element serving as “s” and the element serving as “o” have the relation indicated by the element serving as “r” depends on how accurately the knowledge graph indicates the relation between the individual elements. It is considered that as the knowledge graph indicates the relation between the individual elements more accurately, the accuracy with which the user determines, by using the knowledge graph, whether the element serving as “s” and the element serving as “o” have the relation indicated by the element serving as “r” tends to increase.

Accordingly, it is desirable to generate a knowledge graph that accurately indicates relations between individual elements. For example, it is desirable to generate a knowledge graph that is formed by arranging nodes representing respective elements in a multidimensional vector space such that relations between the individual elements are accurately indicated by positional relationships between the nodes representing the respective elements. For example, each of the relations is indicated by a vector that couples the nodes.

In relation to this, for example, a method for generating a knowledge graph by using pieces of positive example data as training data such that relations between elements are indicated by positional relationships between nodes representing the respective elements in a multidimensional vector space is conceivable. A piece of positive example data is triple data in which an element serving as “s”, an element serving as “o”, and an element serving as “r” indicating a correct relation between the element serving as “s” and the element serving as “o” are associated with one another.

For example, a knowledge graph is generated such that a score calculated for each piece of positive example data by a predetermined score function defined for the knowledge graph becomes high as a whole. The score is an index value that increases as the relation, which is indicated by the data, between the element serving as “s” and the element serving as “o” is more appropriate. For example, a knowledge graph is generated such that the index value is optimized by using a cost function for calculating the index value that indicates the overall height of the score calculated for the pieces of positive example data. The index value is, for example, a total value, a minimum value, a maximum value, an average value, a mode value, a median value, or the like. For example, if the index value is a total value, the optimization corresponds to maximization.

For example, a method for generating a knowledge graph by further using pieces of negative example data as training data such that relations between elements are indicated by positional relationships between nodes representing the respective elements in the multidimensional vector space is also conceivable. A piece of negative example data is triple data in which an element serving as “s”, an element serving as “o”, and an element serving as “r” indicating an incorrect relation between the element serving as “s” and the element serving as “o” are associated with one another.

For example, a knowledge graph is generated such that a score calculated for each piece of positive example data by a predetermined score function is high as a whole and such that a score calculated for each piece of negative example data is low as a whole. For example, a knowledge graph is generated such that the index value is optimized by using a cost function for calculating the index value that comprehensively indicates the overall height of the score calculated for the pieces of positive example data and the overall lowness of the score calculated for the pieces of negative example data. The index value is, for example, an index value indicating the overall lowness of a difference obtained by subtracting the score calculated for pieces of positive example data corresponding to a certain relation from the score calculated for pieces of negative example data corresponding to the same relation. In this case, the optimization is, for example, minimization.

For a method for generating a knowledge graph, for example, HAYASHI Katsuhiko et al., “Block HolE: Problem of knowledge graph embedding based on simultaneous diagonalization of relation matrix and solution therefor”, IPSI SIG Technical Report (Vol. 2018_SLP_121 No.7), May 2018, pp. 1-8 (hereinafter, referred to as HAYASHI et al.) may be referred to. The predetermined score function is defined in a truth value model for “r” of RESCAL, DistMult, HoIE, ComplEx, Analogy, SimplE, Block HoIE, TransE, TransH, TransR, STransR, or the like.

In the related art, however, a knowledge graph that accurately indicates relations between individual elements may not be generated in some cases. For example, in a case where there are a plurality of relations between certain elements, nodes that represent the respective elements may not be arranged in a multidimensional vector space such that the relations between the individual elements are accurately indicated. Consequently, a knowledge graph that accurately indicates the relations between the elements may not be generated.

For example, in the field of chemistry, there may be a plurality of relations between two proteins A and B in some cases. A case is conceivable where there is a phosphorylation reaction that “the protein A phosphorylates an X-th amino acid (site) of an amino acid sequence of the protein B”. In this case, an element serving as “s” is “A”, an element serving as “o” is “B”, and an element serving as “r” is “X”. In fact, a phenomenon is conceivable in which the protein A causes a phosphorylation reaction at a site s1 of the protein B in the nucleus and causes a phosphorylation reaction at a site s2 of the protein B in the cytoplasm. Each of “s1” and “s2” is an integer indicating the order of the amino acid in the protein B. Thus, the protein B may have a plurality of roles and may have a plurality of relations with the protein A.

By using FIG. 1, description will be given next of what arrangement of nodes representing respective elements in a multidimensional vector space is preferable in a case where individual pieces of positive example data of a positive example data group S are present as training data.

In an example of FIG. 1, the positive example data group S includes pieces of positive example data (ei, rj, ek) such as Data1, Data2, Data3, Data4, Data5, and Data6. Each of the pieces of positive example data (ei, rj, ek) is triple data in which an element ei serving as a subject, an element rj serving as a predicate, and an element ek serving as an object are associated with one another. Each of i, j, and k is a natural number.

When individual vectors indicate different relations in the multidimensional vector space, a larger difference in direction between the individual vectors is more preferable. For example, in a case where individual vectors indicate different relations, it is preferable to arrange nodes representing the respective elements such that the difference in direction between the individual vectors increases. On the other hand, in a case where different vectors indicate an identical relation, it is preferable to arrange nodes representing the respective elements such that the difference in direction between the individual vectors decreases.

First, according to Data1 and Data2, as illustrated in an arrangement example 100, it is considered that arranging a node 101 representing an element e1, a node 102 representing an element e5, and a node 103 representing an element e6 in a multidimensional vector space is preferable. For example, it is preferable that a direction of a vector indicating a relation r1 from the node 101 representing the element e1 to the node 102 representing the element e5 is different from a direction of a vector indicating a relation r2 from the node 101 representing the element e1 to the node 103 representing the element e6.

On the other hand, according to Data3, as illustrated in an arrangement example 110, it is considered that arranging a node 111 representing the element e1 and a node 112 representing the element e6 in the multidimensional vector space is preferable. For example, it is preferable that a direction of a vector indicating a relation r3 from the node 111 representing the element e1 to the node 112 representing the element e6 is different from the direction of the vector indicating the relation r1, from the direction of the vector indicating the relation r2, and the like illustrated in the arrangement example 100.

According to Data4, Data5, and Data6, as illustrated in an arrangement example 120, it is preferable to arrange a node 121 representing an element e2, a node 122 representing an element e3, a node 123 representing the element e5, and a node 124 representing the element e6 in the multidimensional vector space. For example, it is preferable that a direction of a vector indicating the relation rl from the node 121 representing the element e2 to the node 123 representing the element e5 is close to the direction of the vector indicating the relation r1 illustrated in the arrangement example 100.

It is preferable that a direction of a vector indicating the relation r2 from the node 121 representing the element e2 to the node 124 representing the element e6 is close to the direction of the vector indicating the relation r2 illustrated in the arrangement example 100. It is preferable that a direction of a vector indicating the relation r3 from the node 122 representing the element e3 to the node 124 representing the element e6 is close to the direction of the vector indicating the relation r3 illustrated in the arrangement example 110.

Thus, a mismatch occurs between the arrangement of the node 101 representing the element e1 and the node 103 representing the element e6 illustrated in the arrangement example 100 and the arrangement of the node 111 representing the element e1 and the node 112 representing the element e6 illustrated in the arrangement example 110. For this reason, the nodes representing the respective elements may not be arranged in the multidimensional vector space such that the relations between the individual elements are accurately indicated. As a result, it is difficult to optimize the index value calculated by a cost function.

Accordingly, in the present embodiment, a generation method that enables generation of a graph that accurately indicates relations between individual elements will be described. The graph is, for example, a knowledge graph. The description now shifts to description of FIG. 2.

(1-1) An information processing apparatus 200 acquires a plurality of pieces of data. Each of the plurality of pieces of data is triple data in which an element of a first item, an element of a second item, and a relation between the element of the first item and the element of the second item are associated with one another. The first item is, for example, “s” (subject) described above. The second item is, for example, “o” (object) described above. The relation is expressed by, for example, the element serving as “r” (predicate) described above. In the example of FIG. 2, the information processing apparatus 200 acquires, for example, the positive example data group S described above.

(1-2) The information processing apparatus 200 transforms each of identical elements that appear in common as either item of the first item or the second item in two or more pieces of data among the acquired plurality of pieces of data, into a unique element in the plurality of pieces of data. The unique element is, for example, an element that appears just once in the plurality of pieces of data. The transform corresponds to replacement. In the example of FIG. 2, the information processing apparatus 200 transforms, for example, each of the elements e1 that appear in common as “s” in Data1, Data2, and Data3 in the positive example data group S, into a corresponding one of unique elements e11, e12, and e13. The information processing apparatus 200 acquires a positive example data group S′ resulting from the transform.

(1-3) Based on a plurality of pieces of data resulting from the transform, the information processing apparatus 200 generates a knowledge graph that is constituted by a plurality of nodes each of which represents a different element and that indicates a relation between the elements in the multidimensional vector space. In the example of FIG. 2, the information processing apparatus 200 generates a knowledge graph 201. The knowledge graph 201 includes, for example, a node 211 representing the element e11, a node 212 representing the element e12, and a node 213 representing the element e13. The knowledge graph 201 also includes, for example, a node 221 representing the element e2, a node 231 representing the element e3, a node 241 representing the element e5, and a node 251 representing the element e6.

In the knowledge graph 201, for example, the node 211 representing the element e11 and the node 213 representing the element e13 may be arranged in directions different from each other with respect to the node 251 representing the element e6. Thus, the node 211 representing the element e11, the node 213 representing the element e13, and the node 251 representing the element e6 may be arranged such that the difference between the direction of the vector indicating the relation r2 and the direction of the vector indicating the relation r3 increases.

In the knowledge graph 201, the node 211 representing the element e11, the node 212 representing the element e12, and the node 221 representing the element e2, which are determined to have similar properties, for example, may be arranged at close positions. In the knowledge graph 201, the node 213 representing the element e13 and the node 231 representing the element e3, which are determined to have similar properties, for example, may be arranged at close positions. In this manner, by individually taking each property of the plurality of properties of the element e1 into account, which of the other elements has a property similar to the property of the element e1 may be grasped.

The knowledge graph 201 further includes a blank node 210 corresponding to the element e1. The blank node 210 may be coupled to the node 211 representing the element e11, the node 212 representing the element e12, and the node 213 representing the element e13.

In this manner, the information processing apparatus 200 may generate the knowledge graph 201 that accurately indicates the relations between the individual elements. The information processing apparatus 200 may generate the knowledge graph 201 by arranging the nodes representing the respective elements in the multidimensional vector space such that the relations between the individual elements are accurately indicated.

As a result, the information processing apparatus 200 may generate the knowledge graph 201 such that the index value calculated by the cost function is optimized. Thus, the information processing apparatus 200 may generate the knowledge graph 201 that accurately reflects a phenomenon in the real world. The information processing apparatus 200 may allow a user to accurately determine, by using the knowledge graph 201, whether an element serving as “s” and an element serving as “o” have a relation indicated by an element serving as “r”. The information processing apparatus 200 may also allow the user to grasp, by using the knowledge graph 201, which elements have similar properties.

[Example of Information Processing System 300]

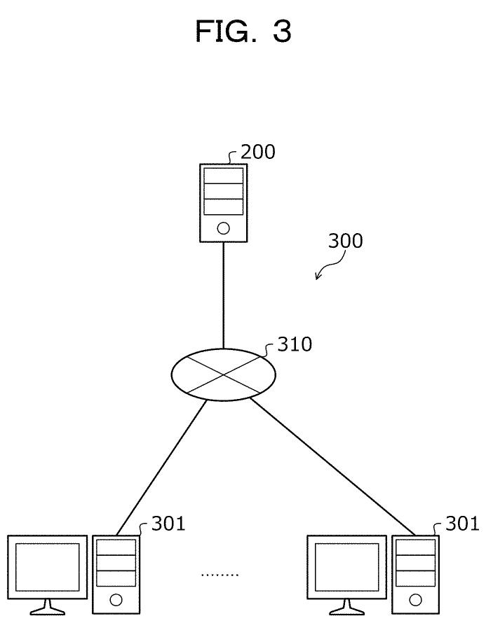

An example of an information processing system 300 to which the information processing apparatus 200 illustrated in FIG. 2 is applied will be described next by using FIG. 3.

FIG. 3 is an explanatory diagram illustrating an example of the information processing system 300. In FIG. 3, the information processing system 300 includes the information processing apparatus 200 and client apparatuses 301.

In the information processing system 300, the information processing apparatus 200 is coupled to the client apparatuses 301 via a network 310 that is wired or wireless. The network 310 is, for example, a local area network (LAN), a wide area network (WAN), Internet, or the like.

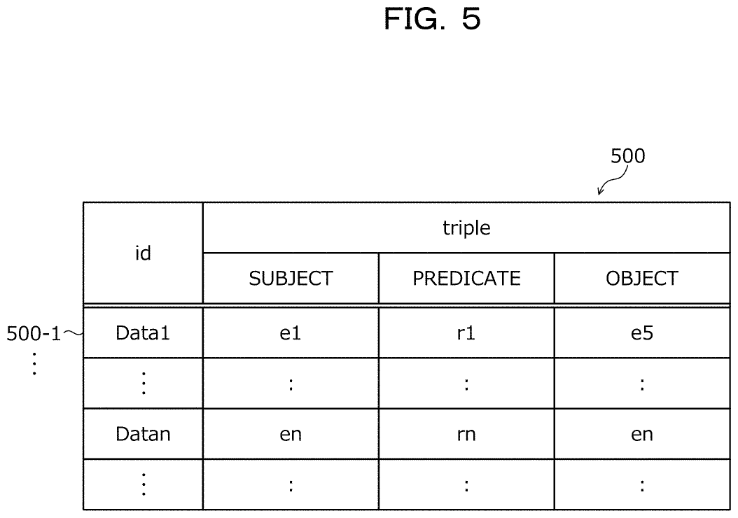

The information processing apparatus 200 is a computer used by an administrator of the information processing system 300. The information processing apparatus 200 stores pieces of positive example data as training data. For example, the information processing apparatus 200 stores the pieces of positive example data in a triple data management table 500 described later in FIG. 5. The information processing apparatus 200 may receive the pieces of positive example data from the client apparatuses 301. The information processing apparatus 200 may further generate and store pieces of negative example data as training data. For example, the information processing apparatus 200 stores the pieces of negative example data in the triple data management table 500 described later in FIG. 5. The information processing apparatus 200 may receive the pieces of negative example data from the client apparatuses 301.

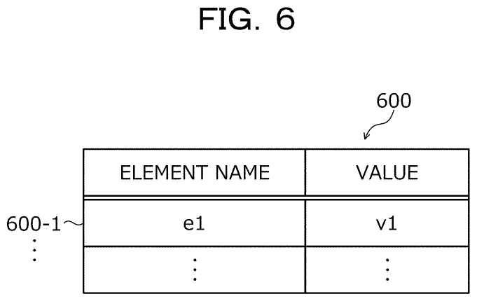

The information processing apparatus 200 generates a knowledge graph. For example, based on the pieces of positive example data, the information processing apparatus 200 generates a knowledge graph. For example, based on the pieces of positive example data and the pieces of negative example data, the information processing apparatus 200 may generate a knowledge graph. The information processing apparatus 200 stores the generated knowledge graph in a knowledge graph management table 600 described later in FIG. 6.

The information processing apparatus 200 provides the client apparatuses 301 with a service in which the generated knowledge graph is used. For example, the information processing apparatus 200 identifies, by using the generated knowledge graph, a possible unknown relation between certain elements. For example, the information processing apparatus 200 transmits information indicating the identified relation to the client apparatuses 301 and allows users of the information processing system 300 to grasp the relation.

For example, the information processing apparatus 200 may receive a request for determining whether there is a predetermined relation between certain elements from each of the client apparatuses 301. In response to the request, for example, the information processing apparatus 200 may transmit, to the client apparatus 301, a result of determining whether there is the predetermined relation between the certain elements by using the generated knowledge graph.

For example, the information processing apparatus 200 may transmit the generated knowledge graph to the client apparatuses 301. The information processing apparatus 200 may identify, by using the generated knowledge graph, which element, among the other elements, has a predetermined relation with a certain element. The information processing apparatus 200 is, for example, a server, a personal computer (PC), or the like.

Each of the client apparatuses 301 is a computer used by a user of the information processing system 300. For example, based on an operation input by a user, the client apparatus 301 may transmit a piece of positive example data to the information processing apparatus 200. For example, based on an operation input by a user, the client apparatus 301 may generate a piece of negative example data from the piece of positive example data, and transmit the generated piece of negative example data to the information processing apparatus 200.

For example, the client apparatus 301 receives, from the information processing apparatus 200, information indicating a possible unknown relation between certain elements, and outputs the information so that the user may grasp the relation. For example, based on an operation input by a user, the client apparatus 301 may transmit a request for determining whether there is a predetermined relation between certain elements to the information processing apparatus 200. For example, the client apparatus 301 receives, from the information processing apparatus 200, a result of determining whether there is the predetermined relation between the certain elements, and outputs the result so that the user may grasp the result.

For example, the client apparatus 301 may receive a knowledge graph from the information processing apparatus 200, and output the knowledge graph so that the user may grasp the knowledge graph. The client apparatus 301 is, for example, a PC, a tablet terminal, a smartphone, or the like.

The description has been given of the case where the information processing apparatus 200 is an apparatus different from the client apparatuses 301. However, the configuration is not limited to this. For example, there may be a case where the information processing apparatus 200 is integral with any of the client apparatuses 301.

[Example of Hardware Configuration of Information Processing Apparatus 200]

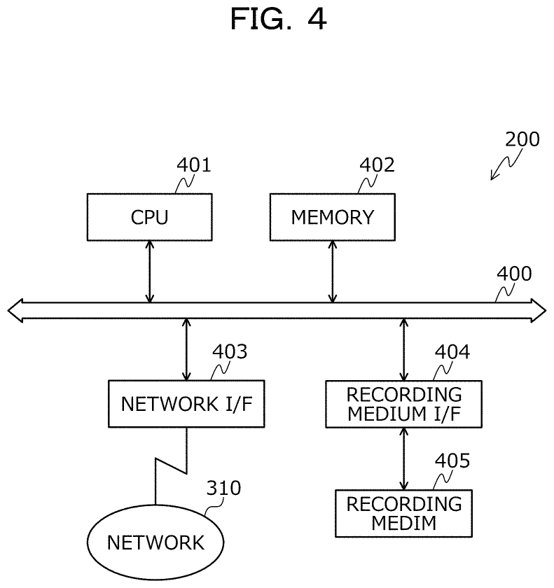

An example of a hardware configuration of the information processing apparatus 200 will be described next by using FIG. 4.

FIG. 4 is a block diagram illustrating an example of a hardware configuration of the information processing apparatus 200. In FIG. 4, the information processing apparatus 200 includes a central processing unit (CPU) 401, a memory 402, a network interface (I/F) 403, a recording medium I/F 404, and a recording medium 405. The individual components are coupled to one another by a bus 400.

The CPU 401 is responsible for controlling the entire information processing apparatus 200. The memory 402 includes, for example, a read-only memory (ROM), a random-access memory (RAM), a flash ROM, and the like. For example, the flash ROM and the ROM store various programs, and the RAM is used as a work area of the CPU 401. A program stored in the memory 402 is loaded by the CPU 401, thereby causing the CPU 401 to perform coded processing.

The network I/F 403 is coupled to the network 310 via a communication line and is coupled to an other computer via the network 310. The network I/F 403 is responsible for an interface between the network 310 and the inside and controls data input to and data output from the other computer. The network I/F 403 is, for example, a modem, a LAN adapter, or the like.

The recording medium I/F 404 controls reading/writing of data from/to the recording medium 405 under the control of the CPU 401. The recording medium I/F 404 is, for example, a port for a disk drive, a solid-state drive (SSD), a Universal Serial Bus (USB), or the like. The recording medium 405 is a nonvolatile memory that stores data written under the control of the recording medium I/F 404. The recording medium 405 is, for example, a disk, a semiconductor memory, a USB memory, or the like. The recording medium 405 may be removably attached to the information processing apparatus 200.

In addition to the components described above, the information processing apparatus 200 may include, for example, a keyboard, a mouse, a display, a printer, a scanner, a microphone, a speaker, or the like. The information processing apparatus 200 may include a plurality of recording medium I/Fs 404 and a plurality of recording media 405. The recording medium I/F 404 and the recording medium 405 may be omitted from the information processing apparatus 200.

[Stored Content of Triple Data Management Table 500]

An example of stored content of the triple data management table 500 will be described next by using FIG. 5. The triple data management table 500 is, for example, implemented by a storage area of the memory 402, the recording medium 405, or the like of the information processing apparatus 200 illustrated in FIG. 4.

FIG. 5 is an explanatory diagram illustrating an example of stored content of the triple data management table 500. As illustrated in FIG. 5, the triple data management table 500 has fields “id”, “subject”, “predicate”, and “object”. In the triple data management table 500, information is set in each field for each piece of triple data, so that the piece of triple data is stored as a record 500-a. “a” is any integer.

In the field “id”, an identifier (id) for identifying a piece of triple data that is a piece of positive example data or a piece of negative example data is set. An element serving as the subject is set in the field “subject”. An element serving as the predicate is set in the field “predicate”. An element serving as the object is set in the field “object”.

[Stored Content of Knowledge Graph Management Table 600]

An example of stored content of the knowledge graph management table 600 will be described next by using FIG. 6. The knowledge graph management table 600 is, for example, implemented by a storage area of the memory 402, the recording medium 405, or the like of the information processing apparatus 200 illustrated in FIG. 4.

FIG. 6 is an explanatory diagram illustrating an example of stored content of the knowledge graph management table 600. As illustrated in FIG. 6, the knowledge graph management table 600 has fields “element name” and “value”. In the knowledge graph management table 600, information is set in each field for each element, so that knowledge graph information is stored as a record 600-b. “b” is any integer.

In the field “element name”, an element name for identifying any element among an element serving as the subject, an element serving as the predicate, and an element serving as the object is set. In the field “value”, a vector or matrix representing an element identified by the element name in the multidimensional vector space is set.

[Example of Hardware Configuration of Client Apparatus 301]

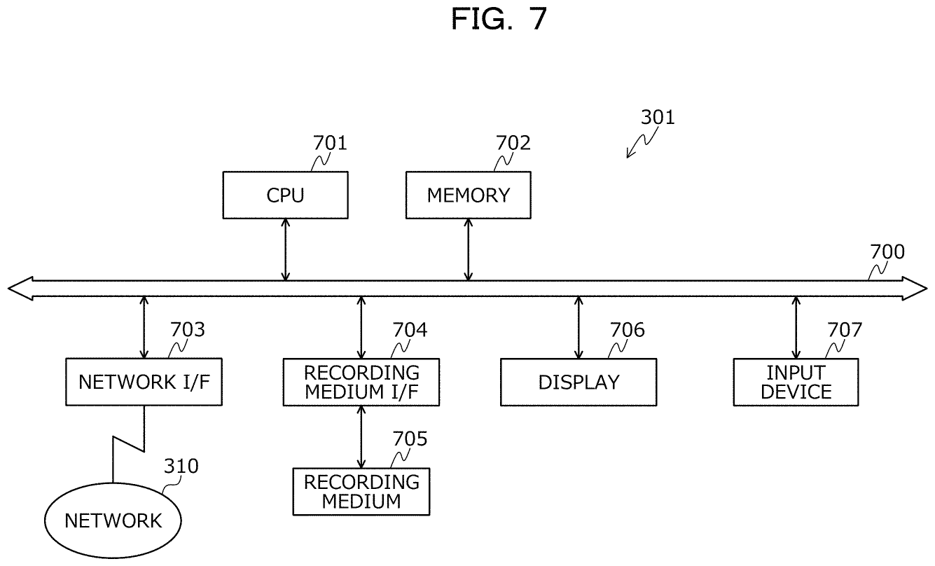

An example of a hardware configuration of each of the client apparatuses 301 included in the information processing system 300 illustrated in FIG. 2 will be described next by using FIG. 7.

FIG. 7 is a block diagram illustrating an example of a hardware configuration of the client apparatus 301. In FIG. 7, the client apparatus 301 includes a CPU 701, a memory 702, a network I/F 703, a recording medium I/F 704, a recording medium 705, a display 706, and an input device 707. The individual components are coupled to one another by a bus 700.

The CPU 701 is responsible for controlling the entire client apparatus 301. The memory 702 includes, for example, a ROM, a RAM, a flash ROM, and the like. For example, the flash ROM and the ROM store various programs, and the RAM is used as a work area of the CPU 701. A program stored in the memory 702 is loaded by the CPU 701, thereby causing the CPU 701 to perform coded processing.

The network I/F 703 is coupled to the network 310 via a communication line and is coupled to an other computer via the network 310. The network I/F 703 is responsible for an interface between the network 310 and the inside and controls data input to and data output from the other computer. The network I/F 703 is, for example, a modem, a LAN adapter, or the like.

The recording medium I/F 704 controls reading/writing of data from/to the recording medium 705 under the control of the CPU 701. The recording medium I/F 704 is, for example, a port for a disk drive, an SSD, a USB, or the like. The recording medium 705 is a nonvolatile memory that stores data written under the control of the recording medium I/F 704. The recording medium 705 is, for example, a disk, a semiconductor memory, a USB memory, or the like. The recording medium 705 may be removably attached to the client apparatus 301.

The display 706 displays a cursor, an icon, a tool box, and data such as a document, an image, and function information. The display 706 is, for example, a cathode ray tube (CRT) display, a liquid crystal display, an electroluminescence (EL) display, or the like. The input device 707 includes keys for inputting characters, numerals, various instructions, and the like, and inputs data. The input device 707 may be a keyboard, a mouse, or the like or may be a touch-panel-type input pad, numeric keypad, or the like.

In addition to the components described above, the client apparatus 301 may include, for example, a printer, a scanner, a microphone, a speaker, or the like. The client apparatus 301 may include a plurality of recording medium I/Fs 704 and a plurality of recording media 705. The recording medium I/F 704 and the recording medium 705 may be omitted from the client apparatus 301.

[Example of Functional Configuration of Information Processing Apparatus 200]

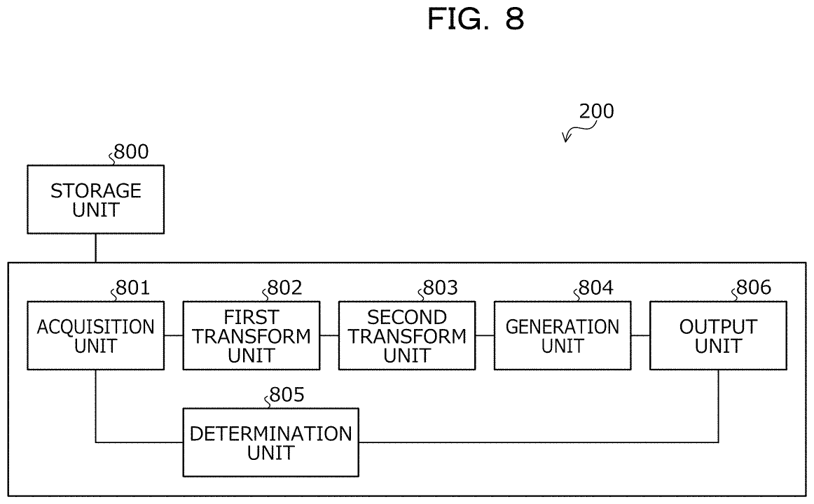

An example of a functional configuration of the information processing apparatus 200 will be described next by using FIG. 8.

FIG. 8 is a block diagram illustrating an example of a functional configuration of the information processing apparatus 200. The information processing apparatus 200 includes a storage unit 800, an acquisition unit 801, a first transform unit 802, a second transform unit 803, a generation unit 804, a determination unit 805, and an output unit 806.

The storage unit 800 is implemented by, for example, a storage area of the memory 402, the recording medium 405, or the like illustrated in FIG. 4. The case where the storage unit 800 is included in the information processing apparatus 200 is described below. However, the configuration is not limited to this. For example, there may be a case where the storage unit 800 is included in an apparatus different from the information processing apparatus 200 and stored content of the storage unit 800 may be referred to from the information processing apparatus 200.

The acquisition unit 801 to the output unit 806 function as an example of a control unit. For example, functions of the acquisition unit 801 to the output unit 806 are implemented by causing the CPU 401 to execute a program stored in the storage area of the memory 402, the recording medium 405, or the like illustrated in FIG. 4 or by using the network I/F 403. A result of processing performed by each of the functional units is stored in, for example, the storage area of the memory 402, the recording medium 405, or the like illustrated in FIG. 4.

The storage unit 800 stores various kinds of information to be referred to or updated in the processing performed by each of the functional units. The storage unit 800 stores training data in which an element of a first item, an element of a second item, and a relation between the element of the first item and the element of the second item are associated with one another. The first item is, for example, a subject. The element of the first item is, for example, an element serving as the subject. The second item is, for example, an object. The element of the second item is, for example, an element serving as the object.

In the training data, for example, the element of the first item, the element of the second item, and an element of a third item indicating the relation between the element of the first item and the element of the second item are associated with one another. The third item is, for example, a predicate. The element of the third item is, for example, an element serving as the predicate. The training data is, for example, triple data. For example, the training data is positive example data or negative example data. The storage unit 800 stores, for example, the triple data management table 500 illustrated in FIG. 5.

The storage unit 800 stores a graph. For example, the graph is generated by the generation unit 804. The graph is, for example, a knowledge graph. In the description below, a case where the generated graph is a “knowledge graph” will be described. A knowledge graph is constituted by a plurality of nodes each representing a different element and indicates relations between the elements in a multidimensional vector space. Each of the nodes is represented by, for example, a vector. The number of dimensions of the multidimensional vector space is set to a relatively large initial value by the administrator, for example. The number of dimensions of the multidimensional vector space is reduced to a value smaller than the initial value, for example, when a knowledge graph is generated.

A relation between elements is indicated by, for example, a positional relationship between nodes corresponding to the respective elements. For example, the relation between the elements is expressed by a vector coupling the nodes corresponding to the elements to each other. For example, the relation between the elements is expressed by a transform vector that enables a vector representing one of the nodes corresponding to the respective elements to be transformed into a vector representing the other of the nodes corresponding to the respective elements by vector addition processing. Thus, the knowledge graph is defined by using, for example, a vector representing each of a plurality of nodes each representing a different element and a vector representing a relation between the elements.

For example, the relation between the elements is expressed by a transform matrix that enables a vector representing one of the nodes corresponding to the respective elements to be transformed into a vector representing the other of the nodes corresponding to the respective elements by matrix multiplication processing. Thus, the knowledge graph is defined by using, for example, a vector representing each of a plurality of nodes each representing a different element and a matrix representing a relation between the elements.

For example, in the field of chemistry, the relation between the elements is a relation between a compound serving as the subject and a chemical property serving as the object and thus is a relation that the compound has the chemical property. For example, in the field of chemistry, the relation between the elements is a relation between a compound serving as the subject and a document serving as the object and thus is a relation that the compound is described in the document. For example, the relation between the elements is a relation between a person serving as the subject and a property serving as the object and thus is a relation that the person has the property. The property is an age, a gender, a belonging organization, an activity time, an activity tendency, or the like. The storage unit 800 stores, for example, the knowledge graph management table 600 illustrated in FIG. 6.

The acquisition unit 801 acquires various kinds of information for use in processing performed by the individual functional units. The acquisition unit 801 stores the acquired various kinds of information in the storage unit 800 or outputs the acquired various kinds of information to the individual functional units. The acquisition unit 801 may output the various kinds of information stored in the storage unit 800 to the individual functional units. For example, based on an operation input by the administrator, the acquisition unit 801 acquires the various kinds of information. For example, the acquisition unit 801 may receive the various kinds of information from an apparatus different from the information processing apparatus 200. For example, the acquisition unit 801 may read the various kinds of information from the recording medium 405 that is removable.

The acquisition unit 801 acquires a plurality of pieces of training data in each of which an element of the first item, an element of the second item, and a relation between the element of the first item and the element of the second item are associated with one another. For example, the acquisition unit 801 acquires a plurality of pieces of positive example data. For example, based on an operation input by the administrator, the acquisition unit 801 acquires a plurality of pieces of positive example data and stores the plurality of pieces of positive example data in the storage unit 800. For example, the acquisition unit 801 may acquire the plurality of pieces of positive example data by receiving the plurality of pieces of positive example data from the client apparatus 301 and store the plurality of pieces of positive example data in the storage unit 800.

The acquisition unit 801 acquires target data. The target data is triple data in which an element of the first item, an element of the second item, and a relation between the element of the first item and the element of the second item are associated with one another. It is determined whether a relation, between the element of the first item and the element of the second item, that is associated with the element of the first item and the element of the second item in the target data is appropriate. For example, based on an operation input by the administrator, the acquisition unit 801 acquires the target data by accepting the input of the target data. For example, the acquisition unit 801 may acquire the target data by receiving the target data from the client apparatus 301.

For example, the acquisition unit 801 may acquire the target data in which, among the plurality of pieces of data, any element of the first item, any element of the second item, and any relation between the element of the first item and the element of the second item are associated with one another by generating the target data. For example, the acquisition unit 801 generates target data in which, among the plurality of pieces of positive example data, any element serving as the subject, any element serving as the object, and any element serving as the predicate indicating the relation are associated with one another.

The acquisition unit 801 may accept a start trigger for starting processing performed by any of the functional units. The start trigger is, for example, input of a predetermined operation by the administrator. The start trigger may be, for example, receipt of predetermined information from an other computer. The start trigger may be, for example, output of predetermined information by any of the functional units.

The acquisition unit 801 may accept a predetermined operation input by the administrator as the start trigger for starting processing performed by the first transform unit 802, the second transform unit 803, and the generation unit 804. The acquisition unit 801 may accept acquisition of a plurality of pieces of training data as the start trigger for starting the processing performed by the first transform unit 802, the second transform unit 803, and the generation unit 804. The acquisition unit 801 may accept acquisition of the target data as the start trigger for starting processing performed by the determination unit 805.

The first transform unit 802 transforms each of identical elements that appear in common as either item of the first item or the second item in two or more pieces of data among the acquired plurality of pieces of data, into a unique element in the plurality of pieces of data. For example, the first transform unit 802 transforms each of identical elements that appear as the subject in two or more pieces of positive example data, into a unique element in the plurality of pieces of positive example data.

For example, the first transform unit 802 transforms each of the elements e1 that serve as the subject and that appear in common in n pieces of positive example data, into a corresponding one of the unique elements e11, . . . , and e1n. “n” is a natural number of 2 or greater. Consequently, the first transform unit 802 may make it easier to arrange nodes representing respective elements in a multidimensional vector space such that relations between the individual elements are accurately indicated. Thus, the first transform unit 802 may enable generation of a knowledge graph that accurately indicates relations between elements.

The second transform unit 803 classifies each of the unique elements obtained through the transform into one or more element groups by using a distance between nodes representing the respective elements in a case where the plurality of nodes each representing a different element are distributed in the multidimensional vector space based on the plurality of pieces of data resulting from the transform. The element group is, for example, a group of unique elements represented by one or more respective nodes included in a certain range among the nodes representing the respective unique elements obtained through the transform.

For example, based on the plurality of pieces of data resulting from the transform, the second transform unit 803 distributes a plurality of nodes each representing a different element in the multidimensional vector space. For example, by using distances between the nodes representing the respective elements in the case where the nodes are distributed, the second transform unit 803 identifies a node group including one or more nodes included in a certain range among the nodes representing the individual unique elements obtained through the transform. The second transform unit 803 classifies the element represented by each of the nodes included in the identified node group into one element group.

For example, based on the plurality of positive example data resulting from the transform, the second transform unit 803 distributes, in the multidimensional vector space, a plurality of nodes each representing a different element and including nodes representing the unique elements e11, . . . , and e1n. For example, by using the distances between the nodes representing the respective elements in the case of the nodes are distributed, the second transform unit 803 identifies nodes representing the respective elements e11 and e12 and included in a certain range, among the nodes representing the respective unique elements e11, . . . , and e1n. For example, the second transform unit 803 classifies the elements e11 and e12 represented by the identified nodes into an element group “a”.

For example, by using the distances between the nodes representing the respective elements in the case of the nodes are distributed, the second transform unit 803 identifies a node representing the element e13 and included in a certain range among the nodes representing the respective unique elements e11, . . . , and e1n. For example, the second transform unit 803 classifies the element e13 represented by the identified node into an element group “b”. Consequently, the second transform unit 803 may identify an element group in which unique elements that are determined to be able to be treated as the identical element are collected.

The second transform unit 803 retransforms each of the unique elements that are obtained through the transform, that appear in the two or more pieces of data, and that are classified into each element group of the one or more element groups, into an element is different across the element groups and that is identical within the element group. For example, the second transform unit 803 retransforms each of the unique elements classified into the same element group into an element that is different across the element groups and is identical within the element group.

For example, the second transform unit 803 retransforms the elements e11 and e12 classified into the element group “a” into an element Ea that is different across the element groups and is identical within the element group. For example, the second transform unit 803 retransforms the element e13 classified into the element group “b” into an element Eb that is different across the element groups and is identical within the element group. Consequently, the second transform unit 803 may reduce the number of elements while enabling generation of a knowledge graph that accurately indicates relations between the elements.

The generation unit 804 generates a knowledge graph, based on the plurality of pieces of data resulting from the transform. For a method for generating a knowledge graph, for example, HAYASHI et al. cited above may be referred to. For example, the generation unit 804 generates, based on the pieces of positive example data resulting from the transform, a knowledge graph by arranging a plurality of nodes each representing a different element in the multidimensional vector space, and stores the generated knowledge graph in the knowledge graph management table 600 illustrated in FIG. 6.

For example, the generation unit 804 arranges the plurality of nodes each representing a different element in the multidimensional vector space such that the index value given by the cost function for the pieces of positive example data resulting from the transform is optimized. The cost function is, for example, a function for calculating an index value that indicates the overall height of the score calculated for the pieces of positive example data. The optimization is, for example, maximization. For example, the generation unit 804 defines, based on the arrangement result, a knowledge graph by using vectors representing the respective nodes and vectors representing the respective relations between the elements, and stores the knowledge graph in the knowledge graph management table 600 illustrated in FIG. 6. Consequently, the generation unit 804 may generate a knowledge graph that accurately indicates relations between elements.

The generation unit 804 generates a knowledge graph, based on the plurality of pieces of data resulting from the retransform. For a method for generating a knowledge graph, for example, HAYASHI et al. cited above may be referred to. For example, the generation unit 804 generates, based on the pieces of positive example data resulting from the retransform, a knowledge graph by arranging a plurality of nodes each representing a different element in the multidimensional vector space, and stores the generated knowledge graph in the knowledge graph management table 600 illustrated in FIG. 6.

For example, the generation unit 804 arranges the plurality of nodes each representing a different element in the multidimensional vector space such that the index value given by the cost function for the pieces of positive example data resulting from the retransform is optimized. The cost function is, for example, a function for calculating an index value that indicates the overall height of the score calculated for the pieces of positive example data. The optimization is, for example, maximization. For example, the generation unit 804 defines, based on the arrangement result, a knowledge graph by using vectors representing the respective nodes and vectors representing the respective relations between the elements, and stores the knowledge graph in the knowledge graph management table 600 illustrated in FIG. 6. Consequently, the generation unit 804 may generate a knowledge graph that accurately indicates relations between elements.

Based on any data among the plurality of pieces of data resulting from the transform, the generation unit 804 may generate new data by replacing at least any element included in the any data with an other element. The new data is, for example, negative example data. The other element is, for example, any element included in an other piece of data among the plurality of pieces of data resulting from the transform.

For example, the generation unit 804 may generate negative example data by replacing an element that serves as at least either one of the subject or the object and is included in any piece of positive example data among the plurality of pieces of positive example data resulting from the transform, with an other element. For example, the generation unit 804 may generate negative example data by replacing the element e11 serving as the subject and included in any piece of positive example data among the plurality of pieces of positive example data resulting from the transform, with the element e2 serving as the subject or the object and included in an other piece of positive example data. Consequently, the generation unit 804 may increase the amount of training data and may facilitate generation of a knowledge graph that accurately indicates relations between elements.

The generation unit 804 generates a knowledge graph, based on the plurality of pieces of data resulting from the transform and the generated new data. For a method for generating a knowledge graph, for example, HAYASHI et al. cited above may be referred to. For example, the generation unit 804 generates a knowledge graph by arranging a plurality of nodes each representing a different element in a multidimensional vector space, based on the pieces of positive example data resulting from the transform and the pieces of generated negative example data.

For example, the generation unit 804 arranges a plurality of nodes each representing a different element in the multidimensional vector space such that the index value given by the cost function for the pieces of positive example data resulting from the transform and the pieces of generated negative example data is optimized. The cost function is, for example, a function for calculating an index value that comprehensively indicates the overall height of the score calculated for the pieces of positive example data and the overall lowness of the score calculated for the pieces of negative example data. The optimization is, for example, minimization. For example, the generation unit 804 defines, based on the arrangement result, a knowledge graph by using vectors representing the respective nodes and vectors representing the respective relations between the elements, and stores the knowledge graph in the knowledge graph management table 600 illustrated in FIG. 6. Consequently, the generation unit 804 may generate a knowledge graph that accurately indicates relations between elements.

Based on any piece of data among the plurality of pieces of data resulting from the retransform, the generation unit 804 may generate new data by replacing at least any element included in the any piece of data with an other element. The new data is, for example, negative example data. The other element is, for example, any element included in an other piece of data among the plurality of pieces of data resulting from the retransform.

For example, the generation unit 804 may generate negative example data by replacing an element that serves as at least one of the subject or the object and is included in any piece of positive example data among the plurality of pieces of positive example data resulting from the retransform with an other element. For example, the generation unit 804 may generate negative example data by replacing the element Ea that serves as the subject and is included in any piece of positive example data among the plurality of pieces of positive example data resulting from the retransform with the element e2 that serves as the subject or the object included in an other piece of positive example data. Consequently, the generation unit 804 may increase the amount of training data and may facilitate generation of a knowledge graph that accurately indicates relations between elements.

The generation unit 804 generates a knowledge graph, based on the pieces of data resulting from the retransform and the generated new data. For a method for generating a knowledge graph, for example, HAYASHI et al. cited above may be referred to. For example, the generation unit 804 generates a knowledge graph by arranging a plurality of nodes each representing a different element in a multidimensional vector space, based on the pieces of positive example data resulting from the retransform and the pieces of generated negative example data.

For example, the generation unit 804 arranges a plurality of nodes each representing a different element in the multidimensional vector space such that the index value given for the positive example data resulting from the retransform and the generated negative example data by the cost function is optimized. The cost function is, for example, a function for calculating an index value that comprehensively indicates the overall height of the score calculated for the pieces of positive example data and the overall lowness of the score calculated for the pieces of negative example data. The optimization is, for example, minimization. For example, the generation unit 804 defines, based on the arrangement result, a knowledge graph by using vectors representing the respective nodes and vectors representing the respective relations between the elements, and stores the knowledge graph in the knowledge graph management table 600 illustrated in FIG. 6. Consequently, the generation unit 804 may generate a knowledge graph that accurately indicates relations between elements.

The generation unit 804 generates a knowledge graph by using a node representing the original elements before the transform. The original elements before the transform are identical elements that appear in common as either item of the first item or the second item in two or more pieces of data among the plurality of pieces of data. For example, the generation unit 804 generates a knowledge graph by further arranging a blank node representing the original element before the transform that serves as the subject or the object. The generation unit 804 generates a knowledge graph by arranging the blank node representing the original element before the transform serving as the subject or the object such that the black node is coupled to a node representing each of the elements obtained through the transform. Consequently, the generation unit 804 may make it easier to identify the element obtained through the transform from the original element before the transform.

The determination unit 805 determines, by using the generated knowledge graph, whether a relation, indicated by the acquired target data, between the element of the first item and the element of the second item is appropriate. The determination unit 805 calculates a score for the acquired target data by using, for example, a predetermined score function. The score function is a function for calculating a score. The score is, for example, an index value that increases as the relation, indicated by the target data, between the element serving as the subject and the element serving as the object is more appropriate. For example, based on the calculated score, the determination unit 805 determines whether the relation, indicated by the target data, between the element of the first item and the element of the second item is appropriate.

For example, if the calculated score is greater than or equal to a threshold, the determination unit 805 determines that the relation, indicated by the target data, between the element of the first item and the element of the second item is appropriate, and determines that the target data is correct answer data. On the other hand, for example, if the calculated score is less than the threshold value, the determination unit 805 determines that the relation, indicated by the target data, between the element of the first item and the element of the second item is not appropriate, and determines that the target data is not correct answer data. In this manner, the determination unit 805 may determine whether a relation between certain elements is appropriate.

The output unit 806 outputs a processing result obtained by any of the functional units. An output format is, for example, displaying the processing result on a display, outputting the processing result to a printer for printing, transmitting the processing result to an external apparatus through the network I/F 403, or storing the processing result in a storage area of the memory 402, the recording medium 405, or the like. Consequently, the output unit 806 may notify the administrator of the processing result obtained by any of the functional units and improve the convenience of the information processing apparatus 200.

The output unit 806 outputs the generated knowledge graph. For example, the output unit 806 transmits the generated knowledge graph to the client apparatus 301 so that the knowledge graph is displayed on the display 706. Consequently, the output unit 806 may allow the user to grasp the knowledge graph.

The output unit 806 outputs a result of determining whether the relation, indicated by the target data, between the element of the first item and the element of the second item is appropriate. For example, the output unit 806 transmits the result of determining whether the relation, indicated by the target data, between the element of the first item and the element of the second item is appropriate to the client apparatus 301 so that the result is displayed on the display 706. Consequently, the output unit 806 may allow the user to grasp the result of determining whether the relation, indicated by the target data, between the element of the first item and the element of the second item is appropriate.

[One Example of Operation of Information Processing Apparatus 200]

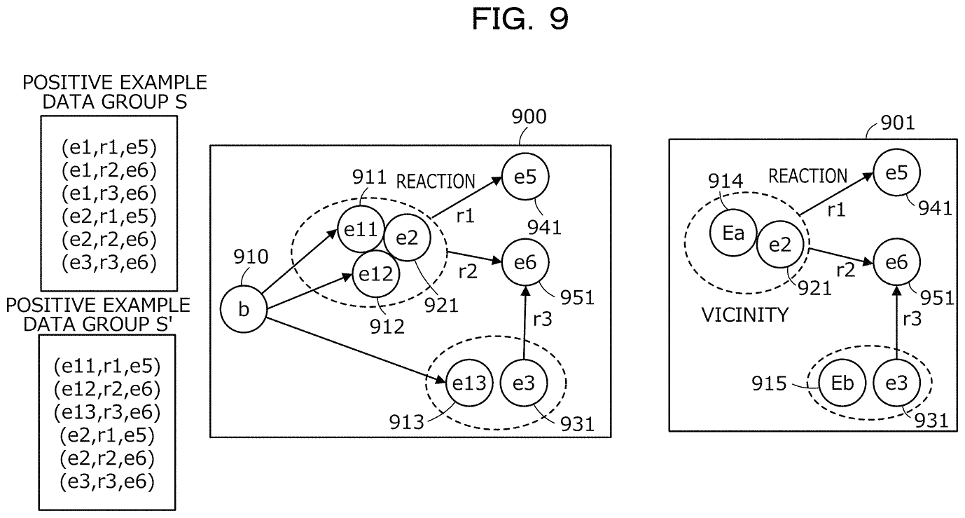

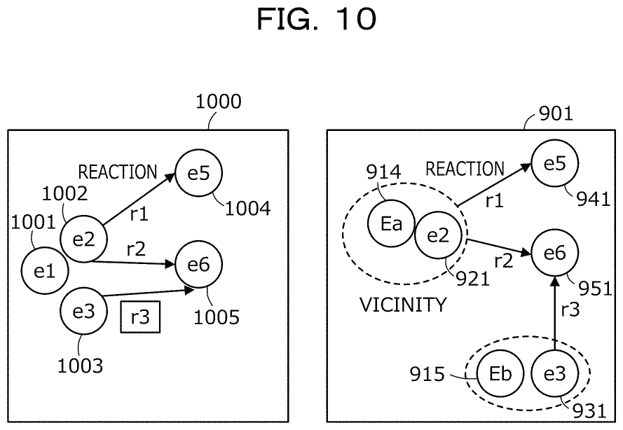

One example of an operation of the information processing apparatus 200 will be described next by using FIGS. 9 and 10.

FIGS. 9 to 10 are explanatory diagrams illustrating one example of an operation of the information processing apparatus 200. In the example of FIG. 9, as in the example of FIG. 1, it is assumed that individual pieces of positive example data of a positive example data group S are present as training data. It is assumed that an entity e1 that appears in common as the subject or the object in the plurality of pieces of positive example data and that is a target of transform is designated by the administrator. An entity is an element. The entity e1 is, for example, an element representing a protein.

In FIG. 9, the information processing apparatus 200 transforms each of the designated entities e1 that appear in common as the subject in Data1, Data2, and Data3 in the positive example data group S, into a corresponding one of unique entities e11, e12, and e13. The information processing apparatus 200 acquires a positive example data group S′ resulting from the transform. Hereinafter, the entities e11, e12, and e13 are treated as elements each representing a different protein.

The information processing apparatus 200 may include, in the positive example data group S′ resulting from the transform, pieces of positive example date in each of which the entity ei that is the target of the transform is associated with each of the unique entities e11, e12, and e13 obtained through the transform.

The information processing apparatus 200 performs embedded learning, based on the positive example data group S′ resulting from the transform. The embedded learning is a process for generating a knowledge graph 900 expressed by using vectors. For convenience of explanation, the knowledge graph 900 is depicted on a two-dimensional plane in the example of FIG. 9 but is not limited to this. For example, the information processing apparatus 200 generates the knowledge graph 900 that is expressed by using vectors, is constituted by a plurality of nodes each representing a different entity, and indicates relations between the entities in a multidimensional vector space.

The knowledge graph 900 includes, for example, a blank node 910 that corresponds to the entity e1. The knowledge graph 900 also includes, for example, a node 911 representing the element e11, a node 912 representing the element e12, and a node 913 representing the element e13. The knowledge graph 900 also includes, for example, a node 921 representing the element e2, a node 931 representing the element e3, a node 941 representing the element e5, and a node 951 representing the element e6.

For example, the information processing apparatus 200 arranges the blank node 910 and the node 911 to 913, 921, 931, 941, and 951 in the multidimensional vector space such that a cost calculated by a predetermined cost function for the positive example data group S′ resulting from the transform is optimized. In this manner, the information processing apparatus 200 expresses the entities e1, e11, e12, e13, e2, e3, e5, and e6 by using vectors.

The cost function is, for example, a function for calculating the total value of values given by a score function. The score function is defined by, for example, Equation (1) below. eₛ is a vector representing an entity that serves the subject, Rᵣ is a matrix representing a relation between entities, and eₒ is a vector representing an entity that serves as the object.

ϕ(s, r, o)=eₛ^{T}Rᵣeₒ (1)

For example, based on the positive example data group S′ resulting from the transform, the information processing apparatus 200 arranges the blank node 910 and expresses the entity e1 as a vector in the multidimensional vector space. For example, based on the positive example data group S′ resulting from the transform, the information processing apparatus 200 arranges the nodes 911, 912, and 913 in the vicinity of the blank node 910 and expresses the entities e11, e12, and e13 as vectors in the multidimensional vector space, respectively. Based on the positive example data group S′ resulting from the transform, the information processing apparatus 200 arranges the nodes 921, 931, 941, and 951 and expresses the entities e2, e3, e5, and e6 as vectors in the multidimensional vector space, respectively.

In this manner, the information processing apparatus 200 may generate the knowledge graph 900 expressed by using vectors such that a difference in direction between vectors indicating different relations increases. The information processing apparatus 200 may generate the knowledge graph 900 expressed by using vectors such that nodes representing entities determined to have similar properties are located closely to each other.

In this way, by individually taking a plurality of properties of the entity e1 into account, the information processing apparatus 200 may generate the knowledge graph 900 expressed by using vectors such that which of the other entities has a property similar to any of the properties of the entity e1 may be grasped. Thus, the information processing apparatus 200 may generate the knowledge graph 900 that is expressed by using vectors and that accurately indicates the relations between the individual entities.

Since the information processing apparatus 200 arranges the blank node 910, the information processing apparatus 200 may enable the knowledge graph 900 to be searched based on the entity e1 and may make it easier to find the entities e11, e12, and e13. Thus, the information processing apparatus 200 may facilitate the use of the knowledge graph 900.

The information processing apparatus 200 classifies the entities e11, e12, and e13 into one or more clusters by using inter-node distances between the nodes 911 to 913, 921, 931, 941, and 951 in the multidimensional vector space. For example, the information processing apparatus 200 classifies the entities e11 and e12 that are at a short distance from each other and are included in a vicinity range into a cluster “a” and classifies the entity e13 having no other close entities into a cluster “b”. In the example of FIG. 9, a range surrounded by a dash line represents the vicinity range.

The information processing apparatus 200 integrates the entities e11 and e12 classified into the cluster “a” into an entity Ea, and arranges a center node 914 representing the entity Ea in the multidimensional vector space. The center node 914 representing the entity Ea is arranged, for example, at an average position of positions of the nodes 911 and 912 representing the entities e11 and e12, respectively.

The information processing apparatus 200 integrates the entity e13 classified into the cluster “b” into an entity Eb, and arranges a center node 915 representing the entity Eb in the multidimensional vector space. The center node 915 representing the entity Eb is arranged, for example, at the same position as the node 913 representing the entity e13. As a result, the information processing apparatus 200 modifies the knowledge graph 900 into a knowledge graph 901.

In this manner, the information processing apparatus 200 may generate the knowledge graph 901 that accurately reflects a phenomenon in the real world. The information processing apparatus 200 may allow the user to accurately determine, by using the knowledge graph 901, whether the entity serving as the subject and the entity serving as the object have the relation indicated by the entity serving as the predicate.

As described above, since the information processing apparatus 200 arranges the center nodes 914 and 915, the information processing apparatus 200 may generate the knowledge graph 901 expressed by using vectors such that how many and what properties the entity e1 has may be grasped. Since the information processing apparatus 200 arranges the blank node 910, the information processing apparatus 200 may make it easier to grasp that the entity el includes the entities e11, e12, and e13.

The case where the information processing apparatus 200 uses Equation (1) described above as the score function has been described. However, the score function is not limited to this. For example, there may be a case where the information processing apparatus 200 uses a score function defined in RESCAL, DistMult, HoIE, ComplEx, Analogy, SimplE, Block HoIE, or the like. For example, there may be a case where the information processing apparatus 200 uses a score function defined in TransE, TransH, TransR, STransR, or the like. The description shifts to description of FIG. 10 next.

In FIG. 10, a case where the knowledge graph 901 generated by the information processing apparatus 200 is compared with a knowledge graph 1000 generated by using a technique of the related art will be described. The knowledge graph 1000 includes, for example, a node 1001 corresponding to the entity e1, a node 1002 representing the element e2, a node 1003 representing the element e3, a node 1004 representing the element e5, and a node 1005 representing the element e6.

In the knowledge graph 1000, the entities e1, e2, e3 each represent a different protein. However, the nodes representing the respective entities e1, e2, and e3 are arranged at positions close to each other. Thus, in the knowledge graph 1000, a difference between directions of vectors indicating the relations r2 and r3 is small. Consequently, the knowledge graph 1000 fails to accurately indicate the relations r2 and r3 in a distinguishable manner. For this reason, in the knowledge graph 1000, the cost calculated by the cost function tends to have a large value.

As a result, even if the user uses the knowledge graph 1000, the user may fail to grasp a possible unknown relation between any entities. For example, even if the user uses the knowledge graph 1000, the user may fail to accurately determine whether any entity that serves as the subject and any entity that serves as the object have a relation indicated by any entity that serves as the predicate.

In contrast, in the knowledge graph 901, the entities e2 and e3 each represent a different protein, and the nodes representing the respective entities e2 and e3 are arranged at positions relatively far from each other. Likewise, in the knowledge graph 901, the entities Ea and Eb represent different properties of the entity el, and the nodes representing the respective entities Ea and Eb are arranged at positions relatively far from each other. Thus, in the knowledge graph 901, a difference between directions of vectors indicating the relations r2 and r3 is large. Consequently, the knowledge graph 901 may accurately indicate the relations r2 and r3 in a distinguishable manner. For this reason, in the knowledge graph 901, the cost calculated by the cost function tends to have a small value.

As a result, the information processing apparatus 200 may enable a possible unknown relation between any entities to be identified by using the knowledge graph 901 and may allow the user to grasp the possible unknown relation between the any entities. For example, the information processing apparatus 200 may accurately determine, by using the knowledge graph 901, whether any entity that serves as the subject and any entity that serves as the object have a relation indicated by any entity that serves as the predicate. The information processing apparatus 200 may allow the user to grasp the determination result. An example in which the information processing apparatus 200 identifies a possible unknown relation between any entities by using the knowledge graph 901 will be described.

For example, based on the knowledge graph 901, the information processing apparatus 200 identifies a plurality of entity pairs that may be formed by combining the entities Ea, Eb, e2, e3, e5, and e6. Based on the knowledge graph 901, the information processing apparatus 200 identifies a plurality of pieces of triple data that may be formed by combining the individual entity pairs with each of the relations r1, r2, and r3 and generates a triple data group. At this time, the information processing apparatus 200 does not have to identify triple data having the same content as any piece of positive example data.

The information processing apparatus 200 determines whether the score calculated for each piece of triple data in the triple data group by the score function D(s, r, o) represented by Equation (1) above is greater than or equal to a threshold. The threshold is equal to, for example, 0.5. If the score for any piece of triple data is greater than or equal to the threshold, the information processing apparatus 200 determines that the relation between the entities indicated by the piece of triple data is “true”. On the other hand, if the score for any piece of triple data is less than the threshold, the information processing apparatus 200 determines that the relation between the entities indicated by the piece of triple data is “false”.

The information processing apparatus 200 transmits the piece of triple data determined to be “true” to the client apparatus 301. In this manner, the information processing apparatus 200 may allow the user to identify a possible unknown relation between any entities.

[Another Example of Operation of Information Processing Apparatus 200]

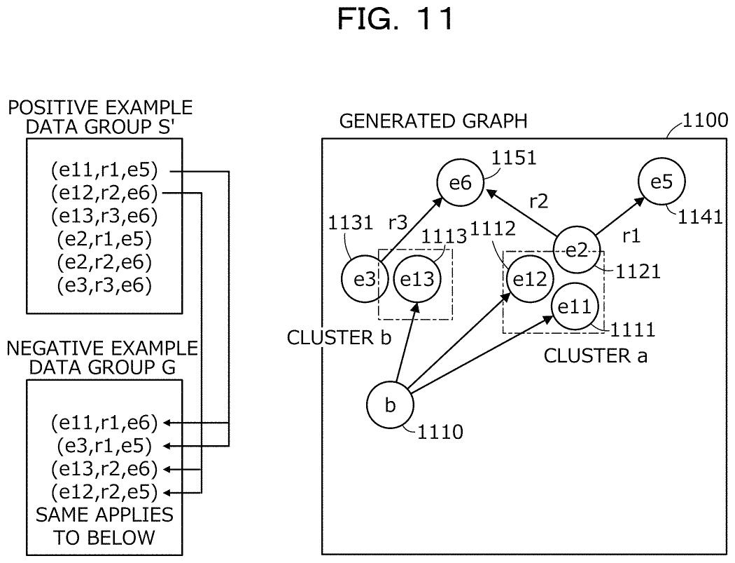

Another example of the operation of the information processing apparatus 200 will be described next by using FIG. 11.

FIG. 11 is an explanatory diagram illustrating another example of the operation of the information processing apparatus 200. It is assumed in FIG. 11 that the information processing apparatus 200 acquires the positive example data group S′ resulting from the transform as in FIG. 9. Based on the positive example data group S′ resulting from the transform and based on pieces of negative example data generated from the respective pieces of positive example data resulting from the transform and included in the positive example data S′ resulting from the transform, the information processing apparatus 200 generates a knowledge graph expressed by using vectors.

For example, the information processing apparatus 200 generates two pieces of negative example data for each piece of positive example data resulting from the transform. Two or more pieces of negative example data may be generated. For example, the information processing apparatus 200 sets an entity group ϵ in which entities that appear in the pieces of positive example data resulting from the transform are collected, and sets a relation group R in which relations that appear in the pieces of positive example data resulting from the transform are collected. In the description below, one piece of positive example data resulting from the transform may be denoted as “(s, r, o)∈S′” by using s, o∈68 and r∈R.

The information processing apparatus 200 generates a piece of negative example data (s′, r, o) obtained by replacing s in the piece of positive example data (s, r, o) resulting from the transform with s′∈ϵ and generates a piece of negative example data (s, r, o′) obtained by replacing o in the piece of positive example data (s, r, o) resulting from the transform with o′∈ϵ. The information processing apparatus 200 generates a negative example data group G in which the pieces of negative example data are collected. In this case, G={(s′, r, o)|′∈ϵ, a(s′, r, o)∈S′}∪{(s, r, o′)|o′∈ϵ, a(s, r, o′)!∈S′} holds. !∈ indicates not including.

The information processing apparatus 200 may generate a piece of negative example data (s′, r, o′) by replacing s and o in the piece of positive example data (s, r, o) resulting from the transform with s′∈ϵ and o′∈ϵ, respectively, and may generate the negative example data group G in which the pieces of negative example data are collected. In this case, G={(s′, r, o′)|s′∈ϵ, o′∈ϵ, a(s′, r, o′)!∈S′} holds.

In the description below, it is assumed that the information processing apparatus 200 generates the piece of negative example data (s′, r, o′) obtained by replacing s and o in the piece of positive example data (s, r, o) resulting from the transform with s′∈ϵ and o′∈ϵ, respectively. Since the information processing apparatus 200 generates the piece of negative example data (s′, r, o′) for each piece of positive example data (s, r, o) resulting from the transform, the information processing apparatus 200 generates one or more pieces of negative example data (s′, r, o′) for each of nᵣ relations included in the positive example data group S′ resulting from the transform.

The information processing apparatus 200 may include, in the positive example data group S′ resulting from the transform, pieces of positive example date in each of which the entity e1 that is the target of the transform is associated with each of the unique entities e11, e12, and e13 obtained through the transform.

The information processing apparatus 200 performs embedded learning, based on the positive example data group S′ resulting from the transform and the negative example data group G. The embedded learning is a process for generating a knowledge graph 1100 expressed by using vectors. For convenience of explanation, the knowledge graph 1100 is depicted on a two-dimensional plane in the example of FIG. 11 but is not limited to this.

For example, the information processing apparatus 200 generates the knowledge graph 1100 that is expressed by using vectors, is constituted by a plurality of nodes each representing a different entity, and indicates relations between the entities in a multidimensional vector space.

The knowledge graph 1100 includes, for example, a blank node 1110 that corresponds to the entity e1. The knowledge graph 1100 also includes, for example, a node 1111 representing the element e11, a node 1112 representing the element e12, and a node 1113 representing the element e13. The knowledge graph 1100 also includes, for example, a node 1121 representing the element e2, a node 1131 representing the element e3, a node 1141 representing the element e5, and a node 1151 representing the element e6.

For example, based on the positive example data group S′ resulting from the transform and the negative example data group G, the information processing apparatus 200 arranges the blank node 1110 and the nodes 1111 to 1113, 1121, 1131, 1141, and 1151 in the multidimensional vector space. At this time, for example, the information processing apparatus 200 optimizes the cost calculated by a predetermined cost function for the positive example data group S′ resulting from the transform and for the negative example data group G. In this manner, the information processing apparatus 200 expresses the entities el, e11, e12, e13, e2, e3, e5, and e6 by using vectors.

The cost function is, for example, a function for calculating an index value based on the score given by the score function for the pieces of positive example data and the score given by the score function for the pieces of negative example data. The score function is defined by, for example, Equation (1) described above. The cost function is defined by, for example, Equation (2) below. L is a cost. [x]₊ is max (0, x). y is a margin hyperparameter. (s, r, o)∈S′ holds. (s′, r, o′)∈G holds. Thus, embedded learning is not to be performed for the combination of the positive example data and the negative example data for which a difference between the score given by the score function for the positive example data and the score given by the score function for the negative example data is greater than or equal to γ.

L=Σ[γ−ϕ(s, r, o)+ϕ(s′, r, o′)] (2)

For example, the information processing apparatus 200 arranges the blank node 1110 and expresses the entity e1 as a vector in the multidimensional vector space. For example, the information processing apparatus 200 arranges the nodes 1111, 1112, and 1113 in the vicinity of the blank node 1110 and expresses the entities e11, e12, and e13 as vectors in the multidimensional vector space. The information processing apparatus 200 arranges the nodes 1121, 1131, 1141, and 1151 and expresses the entities e2, e3, e5, and e6 as vectors in the multidimensional vector space.

In this manner, the information processing apparatus 200 may generate the knowledge graph 1100 expressed by using vectors such that a difference in direction between vectors indicating different relations increases. The information processing apparatus 200 may generate the knowledge graph 1100 expressed by using vectors such that nodes representing entities determined to have similar properties are located closely to each other.

In this way, by individually taking a plurality of properties of the entity e1 into account, the information processing apparatus 200 may generate the knowledge graph 1100 expressed by using vectors such that which of the other entities has a property similar to any of the properties of the entity e1 may be grasped. Thus, the information processing apparatus 200 may generate the knowledge graph 1100 that is expressed by using vectors and that accurately indicates the relations between the individual entities.

Since the information processing apparatus 200 arranges the blank node 1110, the information processing apparatus 200 may enables the knowledge graph 1100 to be searched based on the entity e1 and may make it easier to find the entities e11, e12, and e13. Thus, the information processing apparatus 200 may facilitate the use of the knowledge graph 1100.

The information processing apparatus 200 classifies the entities e11, e12, and e13 into one or more clusters by using inter-node distances between the nodes 1111 to 1113, 1121, 1131, 1141, and 1151 in the multidimensional vector space. For example, the information processing apparatus 200 classifies the entities e11 and e12 that are at a short distance from each other and are included in a vicinity range into a cluster “a” and classifies the entity e13 having no other close entities into a cluster “b”. In the example of FIG. 11, a range surrounded by a dot-dash line represents the vicinity range.

The information processing apparatus 200 integrates the entities e11 and e12 classified into the cluster “a” into the entity Ea, and arranges a center node representing the entity Ea in the multidimensional vector space. In the multidimensional vector space, the center node representing the entity Ea is arranged, for example, at an average position of positions of the nodes 1111 and 1112 representing the entities e11 and e12, respectively.

The information processing apparatus 200 integrates the entity e13 classified into the cluster “b” into the entity Eb, and arranges a center node representing the entity Eb in the multidimensional vector space. In the multidimensional vector space, the center node representing the entity Eb is arranged, for example, at the same position as the node 1113 representing the entity e13.

In this manner, the information processing apparatus 200 may generate the knowledge graph 1100 that accurately reflects a phenomenon in the real world. The information processing apparatus 200 may allow the user to accurately determine, by using the knowledge graph 1100, whether the entity serving as the subject and the entity serving as the object have the relation indicated by the entity serving as the predicate.

As described above, since the information processing apparatus 200 arranges the center nodes representing the entities Ea and Eb, respectively, the information processing apparatus 200 may generate the knowledge graph 1101 expressed by using vectors such that how many and what properties the entity ei has may be grasped. Since the information processing apparatus 200 arranges the blank node 910, the information processing apparatus 200 may make it easier to grasp that the entity ei includes the entities e11, e12, and e13.

Since the information processing apparatus 200 uses the pieces of negative example data, entities having different properties are more likely to be separate from each other or the difference in direction between vectors indicating different relations is more likely to be large in the multidimensional vector space. Thus, the information processing apparatus 200 may generate the knowledge graph 1100 that is expressed by using vectors and that more accurately indicates the relations between the individual entities. For example, the information processing apparatus 200 may generate the knowledge graph 1100 so that the knowledge graph 1100 more accurately reflects a phenomenon in the real world.

Since the information processing apparatus 200 uses the pieces of negative example data, overlearning may be avoided. Thus, the information processing apparatus 200 may allow the user to more accurately determine, by using the knowledge graph 1100, whether the entity serving as the subject and the entity serving as the object have the relation indicated by the entity serving as the predicate.

[Actual Application Example of Information Processing Apparatus 200]

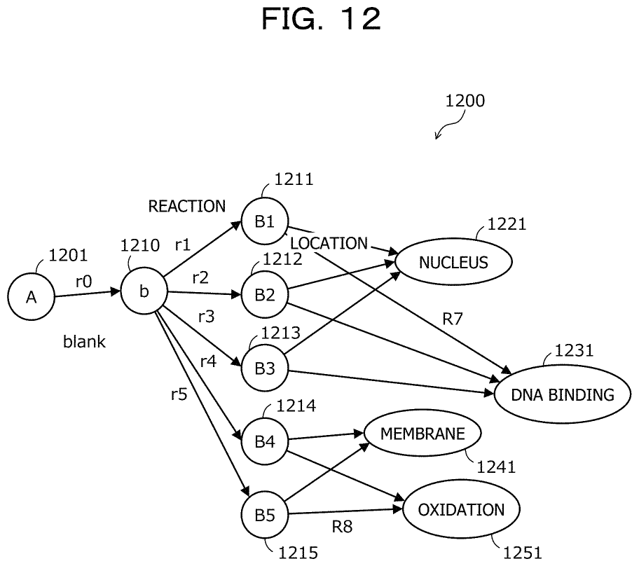

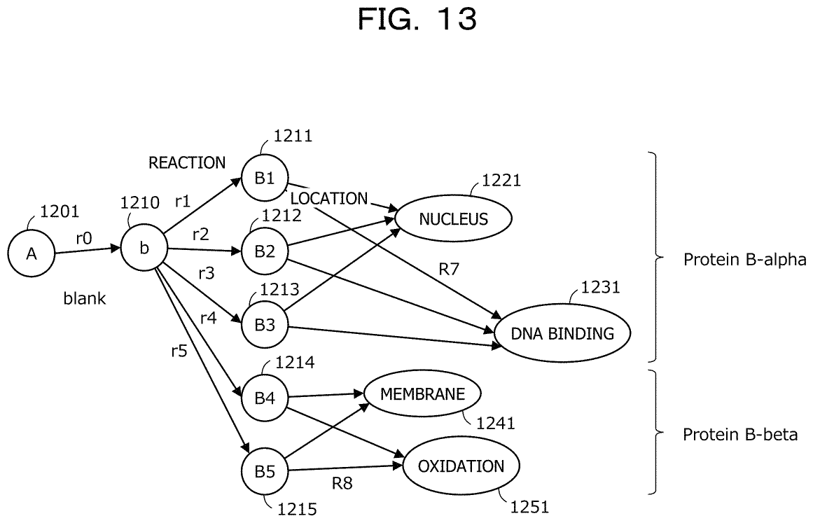

An actual application example of the information processing apparatus 200 will be described next by using FIGS. 12 and 13. The information processing apparatus 200 is used, for example, when a knowledge graph related to the field of chemistry is generated.

FIGS. 12 and 13 are explanatory diagrams illustrating an actual application example of the information processing apparatus 200. In FIG. 12, the information processing apparatus 200 acquires a positive example data group. It is assumed that an entity “A”, an entity “B”, an entity “nucleus”, an entity “DNA binding”, an entity “membrane”, and an entity “oxidation” appear in the positive example data group. The entities “A” and “B” are proteins. It is assumed that the entity “B” is designated as a target of transform by the administrator.

In this case, the information processing apparatus 200 transforms the entity “B” of each piece of positive example data into unique entities B1, B2, B3, B4, and B5, and generates a knowledge graph 1200 expressed by using vectors. The knowledge graph 1200 includes a node 1201 representing the entity “A”. The knowledge graph 1200 also includes nodes 1211, 1212, 1213, 1214, and 1215 representing the entities B1, B2, B3, B4, and B5, respectively.

The knowledge graph 1200 also includes a node 1221 representing the entity “nucleus”, a node 1231 representing the entity “DNA binding”, a node 1241 representing the entity “membrane”, and a node 1251 representing the entity “oxidation”. The knowledge graph 1200 also includes a blank node 1210 corresponding to the entity “B”. Relations between the blank node 1210 and the nodes 1211, 1212, 1213, 1214, and 1215 representing the entity B1, B2, B3, B4, and B5, respectively, are individually defined.

The information processing apparatus 200 randomly arranges the blank node 1210 and the nodes 1211 to 1215, 1221, 1231, 1241, and 1251 in a multidimensional vector space. The information processing apparatus 200 rearranges the blank node 1210 and the nodes 1211 to 1215, 1221, 1231, 1241, and 1251 in the multidimensional vector space such that the cost calculated by a predetermined cost function is optimized. The description now shifts to description of FIG. 13.

As illustrated in FIG. 13, the information processing apparatus 200 automatically arranges various nodes such that the entities B1, B2, B3, B4, and B5 are divided into two groups, based on relations with the entities “nucleus”, “DNA binding”, “membrane”, and “oxidation” in accordance with positional relationships between the various nodes.

The information processing apparatus 200 assigns a name “protein B-a” to the entities B1, B2, and B3 divided into a group, and arranges a center node for the protein B-a in place of the nodes 1211, 1212, and 1213 representing the entities B1, B2, and B3, respectively. The information processing apparatus 200 assigns a name “protein B-13” to the entities B4 and B5 divided into a group, and arranges a center node for the protein B-13 in place of the nodes 1214 and 1215 representing the entities B4 and B5, respectively.

In this manner, the information processing apparatus 200 may generate the knowledge graph 1200 expressed by using vectors such that nodes representing entities determined to have similar properties are located closely to each other in order to temporarily transform certain entities into unique entities. The information processing apparatus 200 may integrate some of the entities obtained through the transform into one entity, and may generate the knowledge graph 1200 that accurately reflects a phenomenon in the real world.

For example, the information processing apparatus 200 may avoid a decrease in accuracy of determining whether a relation between certain entities is appropriate based on the original entities before the integration. The original entities before the integration, which preferably indicate the same element, are treated as different entities. Therefore, determining whether a relation between certain entities is appropriate based on the original entities before the integration is likely to lead to a decrease in accuracy of the determination.

For example, a case where nodes for entities x1 and x2 are present in the vicinity is considered. In this case, when an entity having a predetermined relation with an entity y is to be identified, even if it is correct to identify one entity from among the entities x1 and x2, the other entity is conceivably identified. As a result, the error rate of the determination is increases. In contrast, since the information processing apparatus 200 integrates the entities, the information processing apparatus 200 may avoid a decrease in accuracy of the determination.

[Procedure of Overall Process]

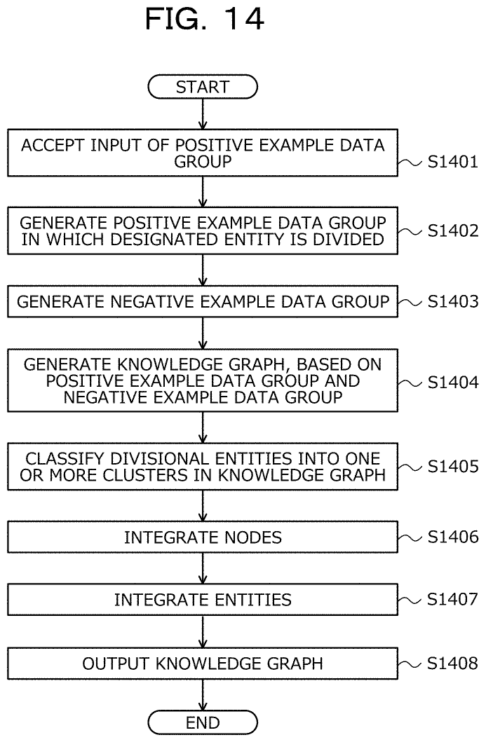

An example of a procedure of an overall process performed by the information processing apparatus 200 will be described next by using FIG. 14. The overall process is implemented by, for example, the CPU 401, the storage area of the memory 402, the recording medium 405, or the like, and the network I/F 403 illustrated in FIG. 4.

FIG. 14 is a flowchart illustrating an example of the procedure of the overall process. In FIG. 14, the information processing apparatus 200 accepts input of a positive example data group as a training data group (operation S1401).

Based on the positive example data group input of which has been accepted, the information processing apparatus 200 generates a positive example data group in which a designated entity is divided (operation S1402). The entity is designated by, for example, the administrator. The entity is, for example, an element that serves as the subject or an element that serves as the object. Based on the generated positive example data group, the information processing apparatus 200 generates a negative example data group (operation S1403).

The information processing apparatus 200 performs embedded learning based on the positive example data group and the negative example data group and generates a knowledge graph expressed by using vectors (operation S1404). The information processing apparatus 200 classifies each of the divisional entities into one or more clusters, based on distances between nodes in the generated knowledge graph (operation S1405).

The information processing apparatus 200 integrates, for each cluster, nodes representing the respective entities classified into the cluster, into one node (operation S1406). The information processing apparatus 200 integrates, for each cluster, the individual entities classified into the cluster, into one entity (operation S1407).

The information processing apparatus 200 outputs the knowledge graph expressed by using vectors (operation S1408). The information processing apparatus 200 then ends the overall process. In this manner, the information processing apparatus 200 may generate the knowledge graph that is expressed by using vectors and that accurately indicates the relations between the individual entities.

[Procedure of Determination Process]

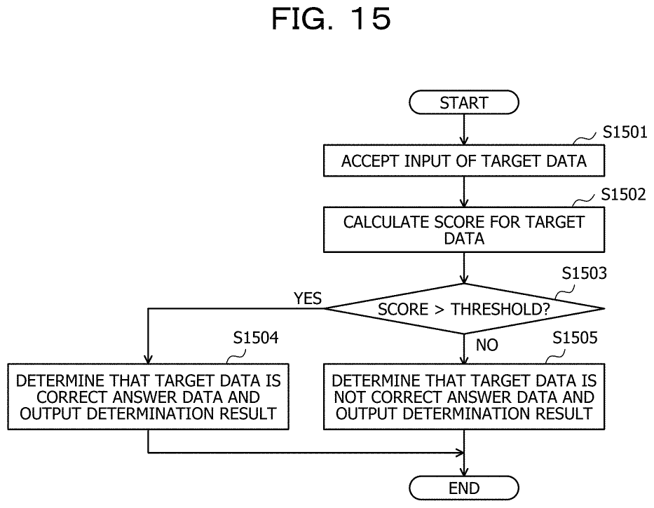

An example of a procedure of a determination process performed by the information processing apparatus 200 will be described next by using FIG. 15. The determination process is implemented by, for example, the CPU 401, the storage area of the memory 402, the recording medium 405, or the like, and the network I/F 403 illustrated in FIG. 4.

FIG. 15 is a flowchart illustrating an example of the procedure of the determination process. In FIG. 15, the information processing apparatus 200 accepts input of target data (operation S1501).

The information processing apparatus 200 calculates, based on the score function, a score for the target data by using the knowledge graph expressed by using vectors (operation S1502). The information processing apparatus 200 determines whether the score is greater than a threshold (operation S1503).

If the score is greater than the threshold (operation S1503: Yes), the information processing apparatus 200 causes the process to proceed to processing in operation S1504. On the other hand, if the score is not greater than the threshold (operation S1503: No), the information processing apparatus 200 causes the process to proceed to processing in operation S1505.

The information processing apparatus 200 determines that the target data is correct answer data, determines that a relation between entities indicated by the target data is “true”, and outputs the determination result (operation S1504). The information processing apparatus 200 then ends the determination process.

The information processing apparatus 200 determines that the target data is not correct answer data, determines that the relation between the entities indicated by the target data is “false”, and outputs the determination result (operation S1505). The information processing apparatus 200 then ends the determination process. In this manner, the information processing apparatus 200 may enable a possible unknown relation between any entities to be identified.

As described above, the information processing apparatus 200 may acquire a plurality of pieces of data in each of which the element of the first item, the element of the second item, and the relation between the element of the first item and the element of the second item are associated with one another. The information processing apparatus 200 may transform each of identical elements that appear in common as either item of the first item or the second item in two or more pieces of data among the acquired plurality of pieces of data, into a unique element in the plurality of pieces of data. Based on the plurality of pieces of data resulting from the transform, the information processing apparatus 200 may generate a graph that is constituted by a plurality of nodes each of which represents a different element and that indicates a relation between the elements in a multidimensional vector space. In this manner, the information processing apparatus 200 may generate the graph that accurately indicates the relations between the individual elements.

The information processing apparatus 200 may classify each of the unique elements obtained through the transform into one or more element groups by using a distance between nodes representing the respective elements in a case where the plurality of nodes each of which represents a different element are distributed in the multidimensional vector space. The information processing apparatus 200 retransforms each of the unique elements that have been classified into each of the one or more groups and that appear in the two or more pieces of data, into an element that is different across the groups and that is identical within the group. The information processing apparatus 200 may generate a graph, based on the plurality of pieces of data resulting from the retransform. In this manner, the information processing apparatus 200 may generate the graph that accurately reflects a phenomenon in the real world. The information processing apparatus 200 may enable whether a possible unknown relation between certain entities is appropriate to be accurately determined by using the graph.

Based on any piece of data among the plurality of pieces of data resulting from the transform, the information processing apparatus 200 may generate new data that is obtained by replacing at least any element included in the any piece of data with an other element. The information processing apparatus 200 may generate the graph, based on the plurality of pieces of data resulting from the transform and the generated new data. In this manner, the information processing apparatus 200 may make it easier to generate the graph that more accurately indicates the relations between the individual elements.

The information processing apparatus 200 may acquire target data in which an element of the first item, an element of the second item, and a relation between the element of the first item and the element of the second item are associated with one another. The information processing apparatus 200 may determine, by using the generated graph, whether the relation, indicated by the acquired target data, between the element of the first item and the element of the second item is appropriate. In this manner, the information processing apparatus 200 may accurately determine whether a possible unknown relation between certain entities is appropriate by using the graph.

The information processing apparatus 200 may generate the target data in which, among the plurality of pieces of data, any element of the first item, any element of the second item, and any relation between the element of the first item and the element of the second item are associated with one another. In this manner, the information processing apparatus 200 may generate a target for which whether a possible unknown relation between any entities is appropriate is to be determined. The information processing apparatus 200 may allow the user to grasp whether there may be an appropriate relation between any entities.

The information processing apparatus 200 may generate the graph by using a node that represents the original elements before the transform. In this manner, the information processing apparatus 200 may make the graph easier to handle. For example, the information processing apparatus 200 may make it easier to search the graph based on the original elements before the transform.

The information processing apparatus 200 may employ an element serving as the subject as the element of the first item. The information processing apparatus 200 may employ an element serving as the object as the element of the second item. The relation between the element of the first item and the element of the second item is indicated by an element of a third item, and the information processing apparatus 200 may employ an element serving as the predicate as the element of the third item. In this manner, the information processing apparatus 200 may handle triple data in which an element serving as the subject, an element serving as the object, and an element serving as the predicate are associated with one another.

The generation method described in the present embodiment may be implemented as a result of a computer such as a PC or a workstation executing a previously prepared program. The generation program described in the present embodiment is recorded on a computer-readable recording medium and is read from the recording medium and executed by a computer. The recording medium is a hard disk, a flexible disk, a compact disc (CD)-ROM, a magneto optical disk (MO), a Digital Versatile Disc (DVD), or the like. The generation program described in the present embodiment may be distributed via a network, such as the Internet.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a generation program for causing a computer to execute a process, the process comprising:
acquiring a plurality of pieces of data each of which is data in which an element of a first item, an element of a second item, and a relation between the element of the first item and the element of the second item are associated with one another;
transforming each of identical elements that appear in common as either item of the first item or the second item in two or more pieces of data among the acquired plurality of pieces of data, into a unique element in the plurality of pieces of data; and
generating, based on the plurality of pieces of data resulting from the transforming, a graph that is constituted by a plurality of nodes each of which represents a different element and that indicates a relation between elements in a multidimensional vector space.

2. The non-transitory computer-readable recording medium according to claim 1, the process further comprising:
classifying each of unique elements including the unique element obtained through the transforming into one or more groups, by using a distance between the nodes that represent the respective elements, in a case where the plurality of nodes each of which represents a different element are distributed in the multidimensional vector space based on the plurality of pieces of data resulting from the transforming; and
retransforming each of the unique elements that have been classified into each of the one or more groups and that appear in the two or more pieces of data into an element that is different across the groups and is identical within a group of the groups,
wherein the process generates the graph, based on the plurality of pieces of data resulting from the retransforming.

3. The non-transitory computer-readable recording medium according to claim 1, the process further comprising:
generating, based on a piece of data among the plurality of pieces of data resulting from the transforming, new data that is obtained by replacing at least an element included in the piece of data with an other element,
wherein the process generates the graph, based on the plurality of pieces of data resulting from the transforming and the generated new data.

4. The non-transitory computer-readable recording medium according to claim 1, the process further comprising:
acquiring target data in which an element of the first item, an element of the second item, and a relation between the element of the first item and the element of the second item are associated with one another; and
determining, by using the generated graph, whether the relation between the element of the first item and the element of the second item is appropriate.

5. The non-transitory computer-readable recording medium according to claim 4,
wherein, in the acquiring the target data, the target data is generated in which, among the plurality of pieces of data, any element of the first item, any element of the second item, and any relation between the element of the first item and the element of the second item are associated with one another.

6. The non-transitory computer-readable recording medium according to claim 1, wherein the process generates the graph by further using a node that represents the identical elements that have been subjected to the transforming.

7. The non-transitory computer-readable recording medium according to claim 1, wherein
the element of the first item is an element that serves as a subject,
the element of the second item is an element that serves as an object,
the relation between the element of the first item and the element of the second item is indicated by an element of a third item, and
the element of the third item is an element that serves as a predicate.

8. A generation method for causing a computer to execute a process, the process comprising:
acquiring a plurality of pieces of data each of which is data in which an element of a first item, an element of a second item, and a relation between the element of the first item and the element of the second item are associated with one another;
transforming each of identical elements that appear in common as either item of the first item or the second item in two or more pieces of data among the acquired plurality of pieces of data, into a unique element in the plurality of pieces of data; and
generating, based on the plurality of pieces of data resulting from the transforming, a graph that is constituted by a plurality of nodes each of which represents a different element and that indicates a relation between elements in a multidimensional vector space.

9. A generation apparatus comprising:
a memory; and
a processor coupled to the memory and configured to:
acquire a plurality of pieces of data each of which is data in which an element of a first item, an element of a second item, and a relation between the element of the first item and the element of the second item are associated with one another;
transform each of identical elements that appear in common as either item of the first item or the second item in two or more pieces of data among the acquired plurality of pieces of data, into a unique element in the plurality of pieces of data; and
generate, based on the plurality of pieces of data resulting from the transforming, a graph that is constituted by a plurality of nodes each of which represents a different element and that indicates a relation between elements in a multidimensional vector space.
